(19) United States
(12) Patent Application Publication
(10) Pub. No.: US 2018/0169358 A1
(43) Pub. Date: Jun. 21, 2018

(54) Device for Administering CPR to Pets

(71) Applicant: The CPR Lifewrap LLC, Chattanooga, TN (US)

(72) Inventor: Felicia Rena Jackson, Hixson, TN (US)

(21) Appl. No.: 15/886,021

(22) Filed: Feb. 1, 2018

Related U.S. Application Data
(63) Continuation-in-part of application No. 15/091,361, filed on Apr. 5, 2016.
(60) Provisional application No. 62/458,753, filed on Feb. 14, 2017.

(51) Int. Cl. A61M 16/00

(52) U.S. Cl. CPC A61M 16/0048; A61M 2250/00

(57) ABSTRACT

A device for administering cardiopulmonary resuscitation to a mammalian animal of the type having a front leg and a snout. The device includes an overlay configured to overlay a side chest area of the animal, the overlay including a front leg opening for the front leg of the animal, and indicium or indicia or both for guiding the administration of cardiopulmonary resuscitation; and a muzzle configured for positioning over the snout of the animal.

### CROSS REFERENCE TO RELATED APPLICATION

This application claims priority to U.S. Provisional Application No. 62/458,753 filed Feb. 14, 2017, entitled ANIMAL CPR LIFEWRAP, and claims priority to and is a continuation-in-part application of co-pending U.S. patent application Ser. No. 15/091,361 filed Apr. 5, 2016, entitled CPR Template, both incorporated by reference herein in their entireties.

### FIELD

This disclosure relates to the field of equipment for administering cardiopulmonary resuscitation (CPR). More particularly, this disclosure relates to a device for facilitating administration of CPR to mammal pets of the type having four legs and snouts, such as dogs and cats.

### BACKGROUND

Cardiac arrests in pets such as dogs and cats are not uncommon. Cardiac events in pets often result from breathing problems rather than heart issues. Choking, allergic reactions and heat stroke are among the top reasons dogs collapse and need CPR. In many cases, prompt and proper administration of CPR can save the life of a pet that is experiencing cardiac arrest.

However, due to the differences between human anatomy and the anatomy of four-legged animals having snouts, such as dogs and cats, even persons experienced in the administration of CPR to humans may need guidance on administering CPR to a dog or cat.

For example, dogs and cats differ in anatomy from humans in one aspect in that dogs and cats have two locations referred to as the chest, which correspond to the broad sides of the animal between the legs. Humans have a single broad chest area on the front of the body, with narrow sides. Animals must be on their side during administration of CPR, whereas humans must be on their backs.

What is desired is a device that facilitates administration of CPR to a pet or the like.

### SUMMARY

The above and other needs are met by devices for administering cardiopulmonary resuscitation to a mammalian animal of the type having a front leg and a snout.

In one aspect, a device includes an overlay configured to overlay a side chest area of the animal, the overlay including a front leg opening for the front leg of the animal, and indicium or indicia or both for guiding the administration of cardiopulmonary resuscitation; and a muzzle configured for positioning over the snout of the animal.

In another aspect, a device includes an overlay configured to overlay a side chest area of the animal, the overlay including a front leg opening for the front leg of the animal, a closure associated with the front leg opening to releasably maintain the opening closed when installed about the front leg of the animal, and indicium or indicia or both for guiding the administration of cardiopulmonary resuscitation.

The device also includes a muzzle configured for positioning over the snout of the animal. The muzzle includes a cylindrical body configured to surround the snout, with a mesh cap disposed at a distal end of the plastic body so that the distal end of the muzzle is breathable and not a barrier to the travel of air or oxygen to the snout. A fixed length tether connects the muzzle to the overlay and an adjustable length tether adjustably connects the muzzle to the overlay.

Devices according to the disclosure advantageously facilitate the administration of CPR to animals having snouts, such as dogs and cats. The devices provide visual guidance to a rescuer in the administration of chest compressions and rescue breathing. Further, the device serves to enhance the flow of air from the rescuer to the animal, and protect the rescuer from undesirable contact with the animal.

### BRIEF DESCRIPTION OF THE DRAWINGS

Further advantages of the invention are apparent by reference to the detailed description when considered in conjunction with the figures, which are not to scale so as to more clearly show the details, wherein like reference numbers indicate like elements throughout the several views, and wherein:

FIG. 1 shows a device according to the disclosure installed on a dog.

FIG. 2 shows the device of FIG. 1.

FIGS. 3 and 4 depict use of the device of FIG. 1 in the administration of CPR to a dog.

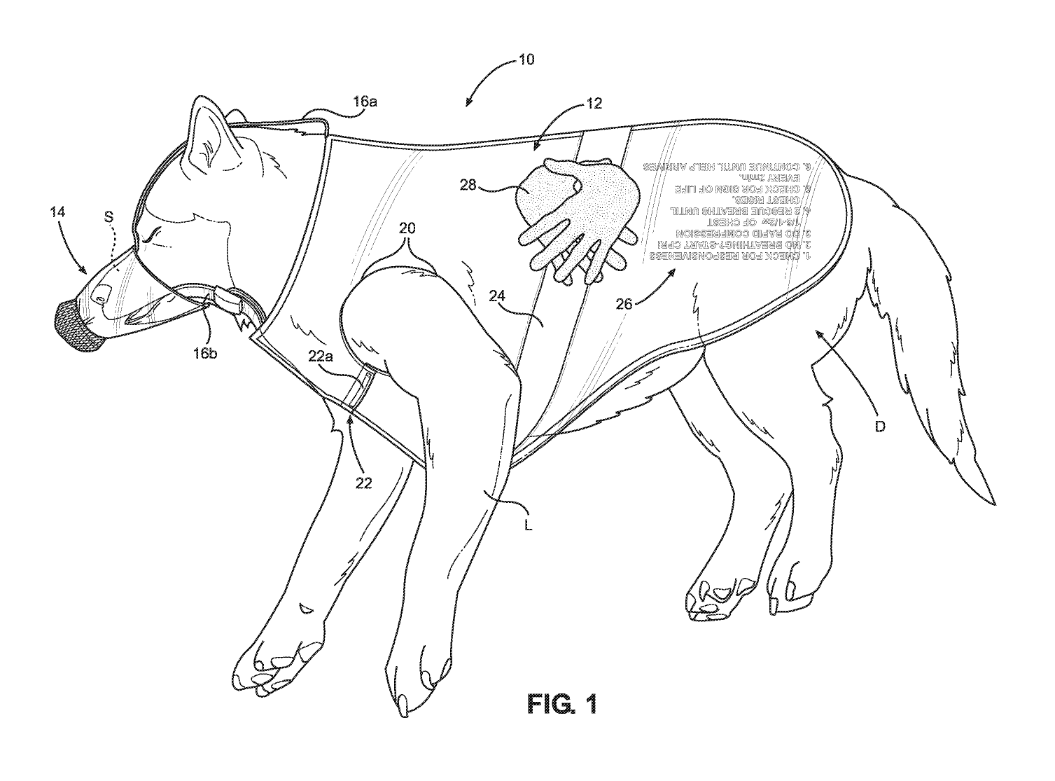

### DETAILED DESCRIPTION

With reference to the drawings, the disclosure relates to a CPR administration device 10 configured to facilitate administration of CPR to pets, such as dogs, cats, and the like having a front leg L and a snout S. The device 10 is preferably disposable, but may be made to be reusable.

The device 10 may be sized to accommodate various sizes of animals. The device 10 as shown in configured for an animal, such as a medium size dog D. The device 10 is also preferably provided in a larger size for larger dogs, and in a smaller size for small dogs and cats. A pet owner or the like may keep on hand one or more of the devices corresponding to the animals present or likely to be present.

The CPR administration device 10 includes an overlay 12 and a muzzle 14 tethered to the overlay 12 as by straps 16a and 16b.

During use of the device 10 to administer CPR to an animal such as the dog D, the dog D is positioned to lie on either side, and the overlay 12 is laid over a chest area of the exposed side of the dog, and the muzzle 14 is applied over the snout S of the dog. The straps 16a and 16b aid in maintaining the muzzle 14 as a unit with the overlay 12, and installed onto the snout S of the dog.

The overlay 12 is of one-piece flexible construction, preferably made of a thermoplastic polyurethane (TPU) film and configured to generally conform to the side or chest area of the dog D. The TPU film is preferred because it is printable and opaque to permit the user to both see instructions and other indicia printed thereon and to permit visual inspection of the animal therethrough, such as to view whether the chest is rising and falling to indicate breathing of the dog. The overlay 12 is configured to extend from a neck of the dog D to a rear hip, and enlarges in dimension adjacent a front shoulder area of the dog.

The overlay 12 includes a front leg opening or aperture 20 configured to receive the front leg L of the dog D. To facilitate installation of the front leg aperture 20 onto the leg L, a slit 22 extends from adjacent edge 12a of the overlay 12. The slit 22 preferably includes a closure such as opposed hook/loop material or opposite charged magnets 22a on the opposing sides of the slit 22 to releasably maintain the slit 22 closed to retain the leg aperture 20 installed on the leg L. A lower surface of the overlay preferably includes a strip of frictional material 24, such as rubber or the like to inhibit slippage of the overlay 12. The frictional material 24 is desirably oriented to overlay a lung area of the dog, where chest compressions are to be applied during the administration of CPR.

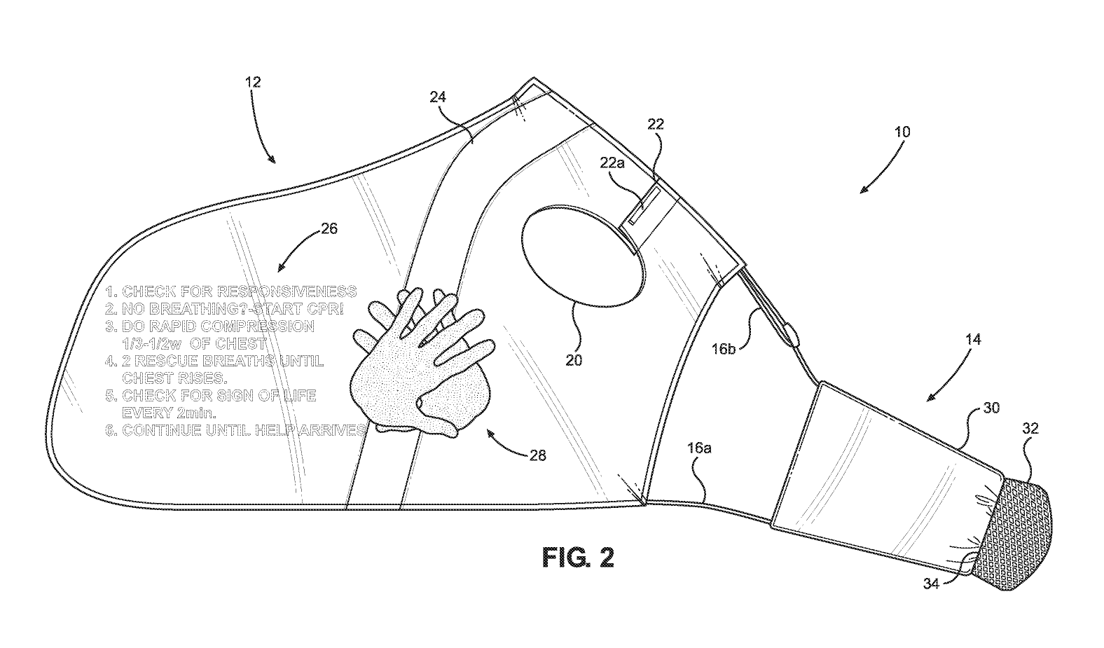

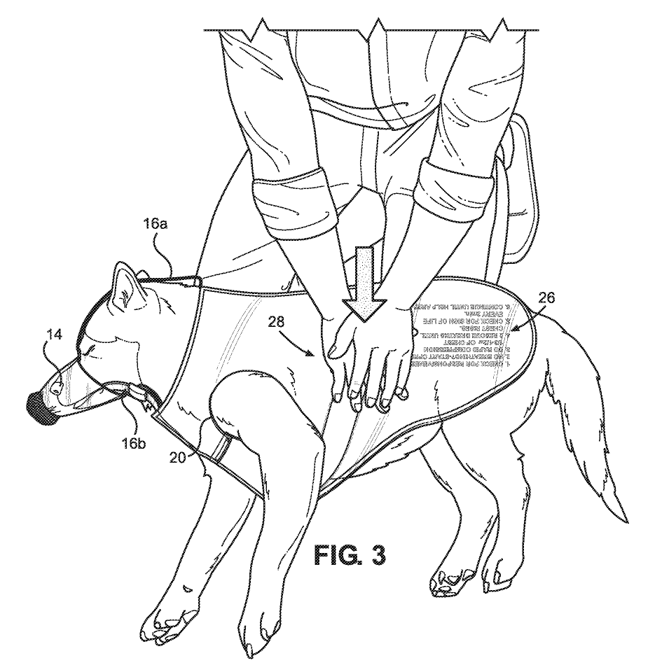

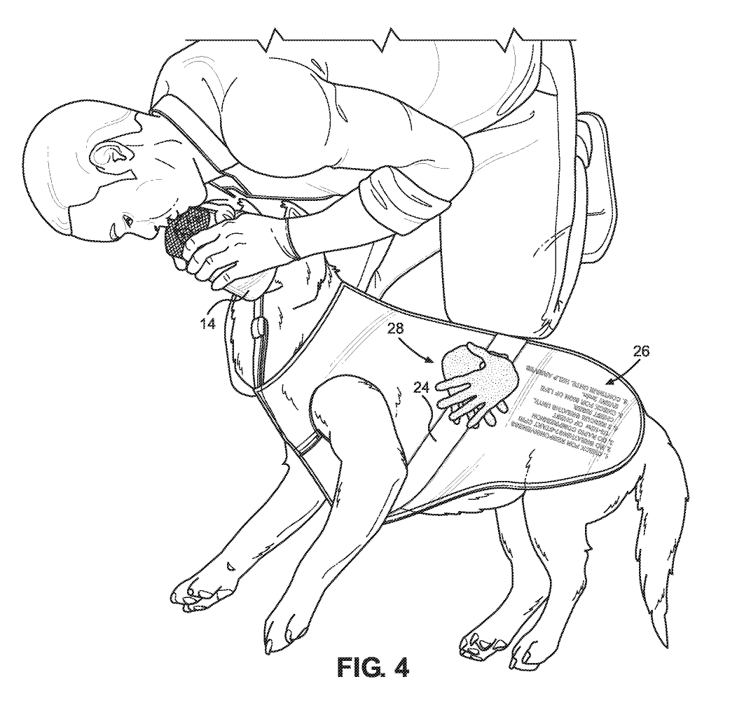

To facilitate the administration of CPR, the overlay includes printed indicia 26 in the nature of CPR instructions and printed indicium 28 in the nature of desired hand position for administration of chest compression. Desired instructions corresponding to the indicia 26 is shown in FIG. 2, for example, the indicia 26 is oriented so that the person administering CPR can read the instructions while administering CPR. This is seen, for example, in FIG. 3. The indicium 28 is oriented to indicate correct placement of the hands during administration of chest compressions, as also shown in FIG. 3.

The muzzle 14 functions as a protective barrier during mouth to snout rescue breathing. The muzzle 14 is configured to facilitate mouth to snout breathing, to protect against direct contact between the hands and the mouth of the rescuer and the snout S, and also serves as a conventional muzzle to protect the rescuer from the teeth of the dog in the event the dog is revived and becomes aggressive.

The muzzle 14 has a cylindrical soft plastic body 30 configured to surround the snout S, with a mesh cap 32 disposed at the distal end of the plastic body 30 so that the end of the muzzle 14 is breathable or otherwise not a barrier to the travel of air or oxygen to the snout S. To prevent slippage, an elastic band 34 is preferably located between the body 30 and the cap 32 to snugly conform to the snout.

The strap 16a is preferably of fixed length and tethers the muzzle 14 to the overlay 12 and maintains it proximate the head of the dog for quick application. The strap 16b is preferably of adjustable length to enable the muzzle 14 to be snugly maintained in place. Thus, once the muzzle 14 is installed, the strap 16b may be adjusted so that a snug relationship between the muzzle 14 and the overlay 12 is maintained. The straps 16a and 16b may be provided of various elongate materials, including rope, strapping, elastic cordage, bands, and the like.

CPR administration devices according to the disclosure advantageously facilitate the administration of CPR to animals having snouts, such as dogs and cats. The devices are quickly and easily installed on an animal. Once installed on an animal, the devices provide visual guidance to a rescuer in the administration of chest compressions and rescue breathing. Further, the device serves to enhance the flow of air from the rescuer to the animal, and protect the rescuer from undesirable contact with the animal.

The foregoing description of embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A device for administering cardiopulmonary resuscitation to a mammalian animal of the type having a front leg and a snout, the device comprising:
an overlay configured to overlay a side chest area of the animal, the overlay including a front leg opening for the front leg of the animal, and indicium or indicia or both for guiding the administration of cardiopulmonary resuscitation; and
a muzzle configured for positioning over the snout of the animal.

2. The device of claim 1, wherein the overlay is positionable on the chest area of the animal to extend from a neck of the dog to a rear hip, and enlarges in dimension adjacent a front shoulder area of the animal.

3. The device of claim 1, wherein the overlay includes a slit extending from an edge of the overlay to the front leg opening to facilitate installation of the leg opening about the front leg of the animal.

4. The device of claim 3, further comprising a closure associated with the slit to releasably maintain the slit closed when installed about the front leg of the animal.

5. The device of claim 4, wherein the closure comprises magnets.

6. The device of claim 1, wherein the muzzle comprises a cylindrical soft body configured to surround the snout, with a mesh cap disposed at a distal end of the plastic body so that the distal end of the muzzle is breathable and not a barrier to the travel of air or oxygen to the snout.

7. The device of claim 1, wherein the muzzle is tethered to the overlay.

8. The device of claim 7, wherein the tether comprises a fixed length tether and an adjustable length tether.

9. A device for administering cardiopulmonary resuscitation to a mammalian animal of the type having a front leg and a snout, the device comprising:
an overlay configured to overlay a side chest area of the animal, the overlay including a front leg opening for the front leg of the animal, a closure associated with the front leg opening to releasably maintain the opening closed when installed about the front leg of the animal, and indicium or indicia or both for guiding the administration of cardiopulmonary resuscitation;
a muzzle configured for positioning over the snout of the animal, the muzzle comprising a cylindrical body configured to surround the snout, with a mesh cap disposed at a distal end of the plastic body so that the distal end of the muzzle is breathable and not a barrier to the travel of air or oxygen to the snout; and
a fixed length tether connecting the muzzle to the overlay and an adjustable length tether adjustably connecting the muzzle to the overlay.
